(19) United States
(12) Patent Application Publication
(10) Pub. No.: US 2011/0118429 A1
(43) Pub. Date: May 19, 2011

(54) CHARGE TRANSPORT MATERIALS

(75) Inventors: Nora Sabrina Radu, Landenberg, PA (US); Norman A. Herron, Newark, DE (US); Gary Johansson, Hockessin, DE (US)

(73) Assignee: E.I. DU PONT DE NEMOURS AND COMPANY, WILMINGTON, DE (US)

(21) Appl. No.: 11/793,541

(22) PCT Filed: Dec. 23, 2005
(86) PCT No.: PCT/US05/46924

§ 371 Date: Apr. 22, 2008

Related U.S. Application Data
(60) Provisional application No. 60/640,320, filed on Dec. 30, 2004.
(60) Provisional application No. 60/694,913, filed on Jun. 28, 2005.

(51) Int. Cl. C08F 22/30; C07C 261/02; C07C 229/42; C08F 16/34; C08F 26/02

(52) U.S. Cl. 526/298; 560/301; 560/48; 526/315; 526/310

(57) ABSTRACT

Charge transport materials are provided, and methods for making the same.

### CROSS REFERENCE

This application claims benefit to U.S. Provisional Application Ser. Nos. 60/640,320, filed Dec. 30, 2004 and 60/694,913, filed Jun. 28, 2005, the disclosures of which are each incorporated herein by reference in their entireties.

### FIELD

This disclosure relates generally to charge transport materials for example, those found in organic electronic devices, and materials and methods for fabrication of the same.

### BACKGROUND

Organic electronic devices convert electrical energy into radiation, detect signals through electronic processes, convert radiation into electrical energy, or include one or more organic semiconductor layers. Most organic electronic devices include charge transport materials.

Thus, what is needed are additional charge transport materials.

### SUMMARY

In one embodiment, substituted triphenyl-amine compounds and polymers are provided, and methods for making the same, as well as devices and sub-assemblies including the same.

The foregoing general description and the following detailed description are exemplary and explanatory only and are not restrictive of the invention, as defined in the appended claims.

### BRIEF DESCRIPTION OF THE DRAWINGS

Embodiments are illustrated in the accompanying figures to improve understanding of concepts as presented herein.

FIG. 1 is a schematic diagram of an organic electronic device.

The figures are provided by way of example and are not intended to limit the invention. Skilled artisans appreciate that objects in the figures are illustrated for simplicity and clarity and have not necessarily been drawn to scale. For example, the dimensions of some of the objects in the figures may be exaggerated relative to other objects to help to improve understanding of embodiments.

### DETAILED DESCRIPTION

In one embodiment, compounds are provided having formulae I or II:

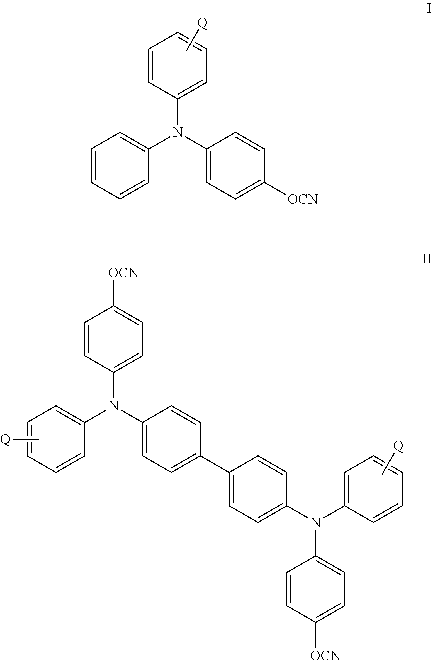

wherein:

Q is, independently at each occurrence, a vinyl group, an acrylate group, or a methacrylate group.

In one embodiment, Q is in the para position.

In one embodiment, Q is a vinyl group.

In one embodiment, a polymer is provided, comprising units formed from at least one of the compounds of formulae I or II. In one embodiment, the polymer is a random, block, graft, or alternating copolymer.

In one embodiment, the polymer is of formula III:

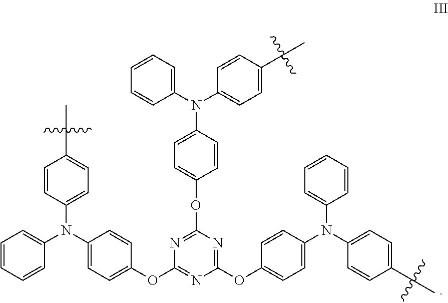

In one embodiment, the polymer is of formula IV:

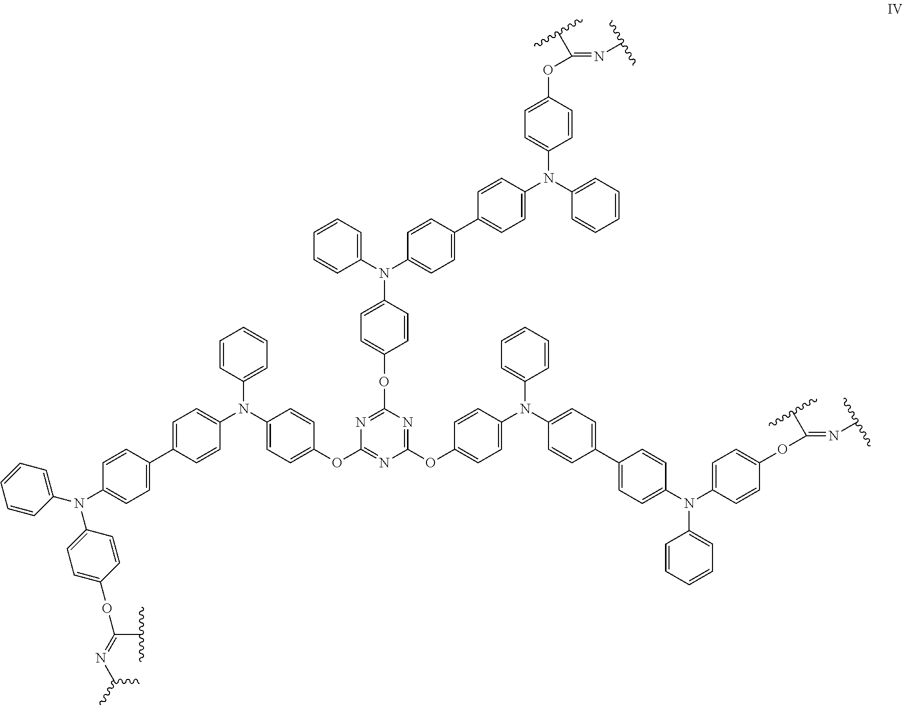

In one embodiment, the present invention comprises a monomer comprising a charge transport group (CTG), and further comprising a first substituent having a polymerizable group, and a second substituent having a reactive group. In one embodiment, the CTG is a triarylamine, triarylmethane, or N-substituted-carbazole. In one embodiment, the polymerizable group is a vinyl group, an acrylate group, or a methacrylate group. In one embodiment, the second substituent has a reactive group. The reactive group is a vinyl group, a cyanate group, a perfluorovinyl ether group, a 3,4-benzocyclobutan-1-yl group, an aldehydic group or a siloxane group. In one embodiment, the reactive group is capable of further reacting to form a polymerizable group.

In one embodiment, the monomer comprises a compound of formula V:

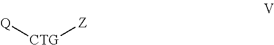

wherein:

CTG is a charge transport group;

Q is a first substituent having a polymerizable group; and

Z is a second substituent having a reactive group.

In another embodiment, the monomer comprises a compound of formula VI:

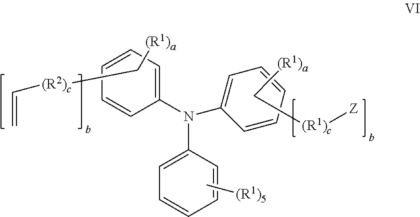

wherein:

R¹ can be the same or different at each occurrence and is selected from H, D, alkyl, heteroalkyl, aryl, heteroaryl, arylalkylene, heteroarylalkylene, CₙH_{d}Fₑ, C₆H_{f}F_{g}, arylamine, arylalkylamine, arylether, alkylether, arythioether and alkylthioether;

R² can be the same or different at each occurrence and is selected from alkyl, heteroalkyl, aryl, heteroaryl, arylalkylene, heteroarylalkylene, CₙHₕFᵢ, C₆HⱼFₖ, arylamine, arylalkylamine, arylether, alkylether, arythioether and alkylthioether;

adjacent R¹ and/or R² can be joined to form a a fused alkyl or aromatic five or six membered ring;

a is 0 or an integer from 1 through 4 and b is an integer from 1 through 5 such that a+b<=5;

C is 0 or an integer from 1 through 20;

d, e, f and g are an integer such that e+f=2n+1−, and g+h=5;

h, i, j and k are an integer such that h+i=2n, and j+k=4;

n is an integer 1 through 20; and

Z is a second substituent having a reactive group.

In one embodiment, the monomer comprises a compound of formula VII or VIII

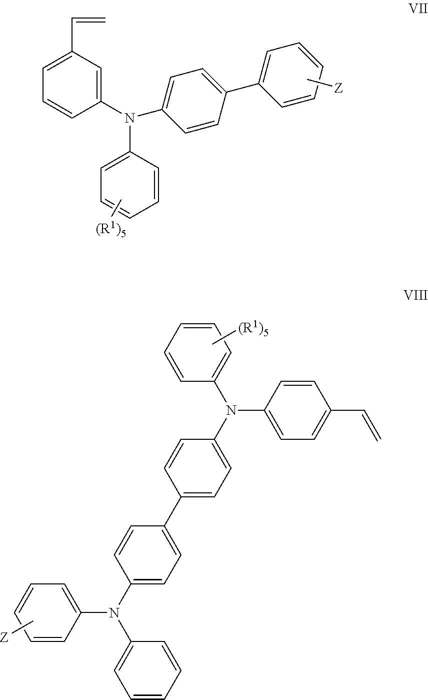

wherein:

R¹ can be the same or different at each occurrence and is selected from H, D, alkyl, heteroalkyl, aryl, heteroaryl, arylalkylene, heteroarylalkylene, CₙH_{d}Fₑ, C₆H_{f}F_{g}, arylamine, arylalkylamine, arylether, alkylether, arythioether and alkylthioether;

adjacent R¹ can be joined to form a fused alkyl or aromatic five or six membered ring;

d, e, f and g are an integer such that e+f=2n+1−, and g+h=5;

n is an integer from 1 through 20; and

Z is a reactive group.

In one embodiment, reactive group is a cyanate ester group. In another embodiment, the reactive group is a perfluorovinyl ether group. In another embodiment, the reactive group is a 3,4-benzocyclobutan-1-yl group. In another embodiment, the reactive group is a siloxane group. In another embodiment, the reactive group can further react to give a polymerizable group and is an aldehyde or a ketone group.

In one embodiment, the invention provides a monomer comprising a compound of formula IX:

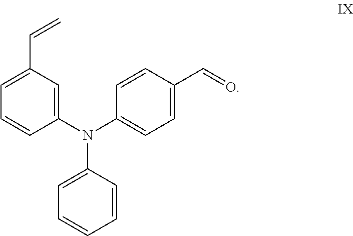

In another embodiment, the invention provides a polymer having at least one monomeric unit derived from a monomer comprising a charge transport group, and further comprising a first substituent having a polymerizable group, and a second substituent having a reactive group.

In another embodiment, the invention provides a polymer comprising at least one monomeric unit derived from a monomer of formula V. In another embodiment, the invention provides a polymer comprising at least one first monomeric unit derived from a monomer of formula V (0-100%) and at least one second monomeric unit derived from a monomer of formula X (0-100%).

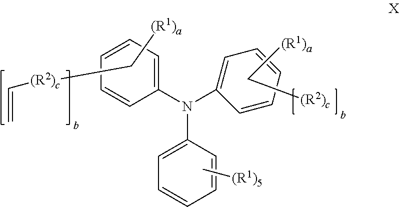

wherein:

R¹ can be the same or different at each occurrence and is selected from H, D, alkyl, heteroalkyl, aryl, heteroaryl, arylalkylene, heteroarylalkylene, CₙH_{d}Fₑ, C₆H_{f}F_{g}, arylamine, arylalkylamine, arylether, alkylether, arythioether and alkylthioether;

R² can be the same or different at each occurrence and is selected from alkyl, heteroalkyl, aryl, heteroaryl, arylalkylene, heteroarylalkylene, CₙHₕFᵢ, C₆HⱼFₖ, arylamine, arylalkylamine, arylether, alkylether, arythioether and alkylthioether;

adjacent R¹ and/or R² can be joined to form a fused alkyl or aromatic five or six membered ring;

a is 0 or an integer from 1 through 4 and b is an integer from 1 through 5 such that a+b<=5;

c is 0 or an integer from 1 through 20;

d, e, f and g are an integer such that e+f=2n+1−, and g+h=5;

h, i, j and k are an integer such that h+i=2n, and j+k=4; and

n is an integer 1 through 20.

In one embodiment, the monomer comprises a compound of formulae XI-XIII

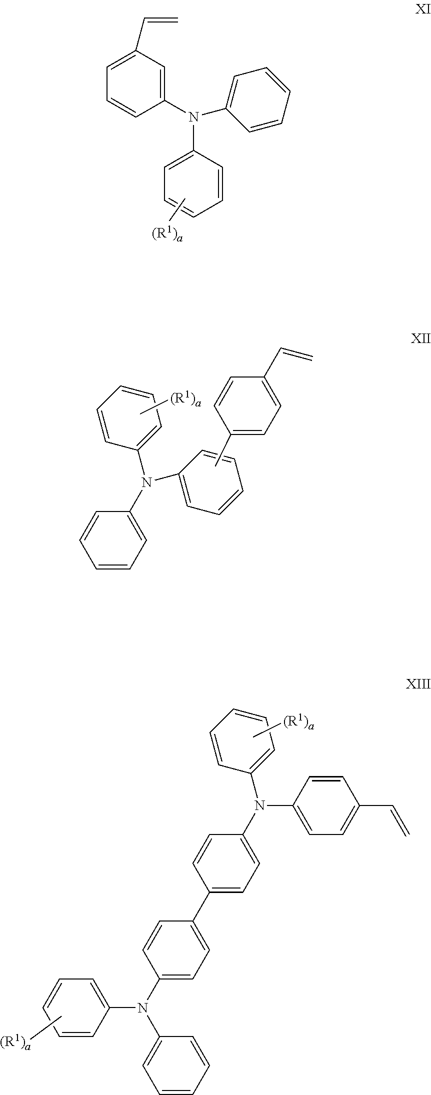

wherein:

R¹ can be the same or different at each occurrence and is selected from H, D, alkyl, heteroalkyl, aryl, heteroaryl, arylalkylene, heteroarylalkylene, CₙH_{d}Fₑ, C₆H_{f}F_{g}, arylamine, arylalkylamine, arylether, alkylether, arythioether and alkylthioether;

adjacent R¹ can be joined to form a fused alkyl or aromatic five or six membered ring;

d, e, f and g are an integer such that e+f=2n+1−, and g+h=5; and

n is an integer 1 through 20.

In one embodiment the invention provides a polymer comprising a compound of formula X having monomeric units derived from monomers of formula XIV, XV, or mixtures thereof:

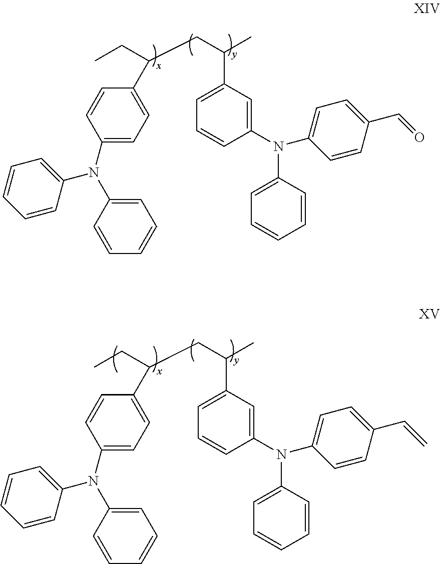

wherein x and y are integers equal to or greater than 1.

In one embodiment, compositions are provided comprising any of the above-described and at least one solvent, processing aid, charge transporting material, or charge blocking material. These compositions can be in any form, including, but not limited to solvents, emulsions, and colloidal dispersions.

### Device

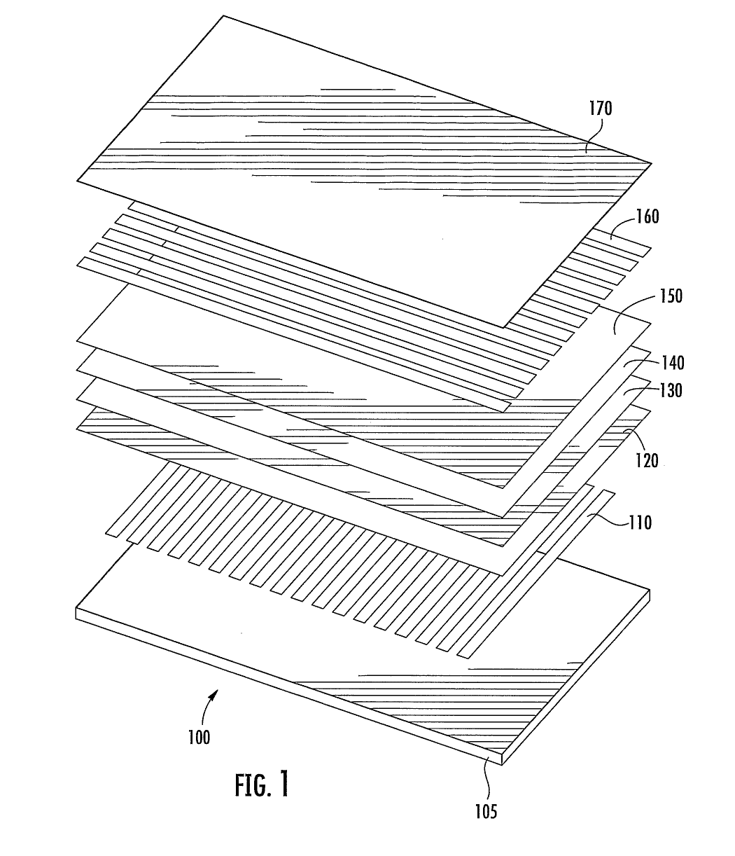

Referring to FIG. 1, an exemplary organic electronic device 100 is shown. The device 100 includes a substrate 105. The substrate 105 may be rigid or flexible, for example, glass, ceramic, metal, or plastic. When voltage is applied, emitted light is visible through the substrate 105.

A first electrical contact layer 110 is deposited on the substrate 105. For illustrative purposes, the layer 110 is an anode layer. Anode layers may be deposited as lines. The anode can be made of, for example, materials containing or comprising metal, mixed metals, alloy, metal oxides or mixed-metal oxide. The anode may comprise a conducting polymer, polymer blend or polymer mixtures. Suitable metals include the Group 11 metals, the metals in Groups 4, 5, and 6, and the Group 8, 10 transition metals. If the anode is to be light-transmitting, mixed-metal oxides of Groups 12, 13 and 14 metals, such as indium-tin-oxide, are generally used. The anode may also comprise an organic material, especially a conducting polymer such as polyaniline, including exemplary materials as described in Flexible Light-Emitting Diodes Made From Soluble Conducting Polymer, Nature 1992, 357, 477-479. At least one of the anode and cathode should be at least partially transparent to allow the generated light to be observed.

An optional buffer layer 120, such as hole transport materials, may be deposited over the anode layer 110, the latter being sometimes referred to as the “hole-injecting contact layer.” Examples of hole transport materials suitable for use as the layer 120 have been summarized, for example, in Kirk Othmer, Encyclopedia of Chemical Technology, Vol. 18, 837-860 (4^{th} ed. 1996). Both hole transporting “small” molecules as well as oligomers and polymers may be used. Hole transporting molecules include, but are not limited to: N,N′ diphenyl-N,N′-bis(3-methylphenyl)-[1,1′-biphenyl]-4,4′-diamine (TPD), 1,1 bis[(di-4-tolylamino) phenyl]cyclohexane (TAPC), N,N′ bis(4-methylphenyl)-N,N′-bis(4-ethylphenyl)-[1,1′-(3,3′-dimethyl)biphenyl]-4,4′-diamine (ETPD), tetrakis (3-methylphenyl)-N,N,N′,N′-2,5-phenylenediamine (PDA), a-phenyl 4-N,N-diphenylaminostyrene (TPS), p (diethylamino)benzaldehyde diphenylhydrazone (DEH), triphenylamine (TPA), bis[4 (N,N-diethylamino)-2-methylphenyl](4-methylphenyl)methane (MPMP), 1 phenyl-3-[p-(diethylamino)styryl]-5-[p-(diethylamino)phenyl]pyrazoline (PPR or DEASP), 1,2 trans-bis(9H-carbazol-9-yl)cyclobutane (DCZB), N,N,N′,N′ tetrakis(4-methylphenyl)-(1,1′-biphenyl)-4,4′-diamine (TTB), and porphyrinic compounds, such as copper phthalocyanine. Useful hole transporting polymers include, but are not limited to, polyvinylcarbazole, (phenylmethyl)polysilane, and polyaniline. Conducting polymers are useful as a class. It is also possible to obtain hole transporting polymers by doping hole transporting moieties, such as those mentioned above, into polymers such as polystyrenes and polycarbonates.

An organic layer 130 may be deposited over the buffer layer 120 when present, or over the first electrical contact layer 110. In some embodiments, the organic layer 130 may be a number of discrete layers comprising a variety of components. Depending upon the application of the device, the organic layer 130 can be a light-emitting layer that is activated by an applied voltage (such as in a light-emitting diode or light-emitting electrochemical cell), or a layer of material that responds to radiant energy and generates a signal with or without an applied bias voltage (such as in a photodetector).

Other layers in the device can be made of any materials which are known to be useful in such layers upon consideration of the function to be served by such layers.

Any organic electroluminescent (“EL”) material can be used as a photoactive material (e.g., in layer 130). Such materials include, but are not limited to, fluorescent dyes, small molecule organic fluorescent compounds, fluorescent and phosphorescent metal complexes, conjugated polymers, and mixtures thereof. Examples of fluorescent dyes include, but are not limited to, pyrene, perylene, rubrene, derivatives thereof, and mixtures thereof. Examples of metal complexes include, but are not limited to, metal chelated oxinoid compounds, such as tris(8-hydroxyquinolato)aluminum (Alq3); cyclometalated iridium and platinum electroluminescent compounds, such as complexes of Iridium with phenylpyridine, phenylquinoline, or phenylpyrimidine ligands as disclosed in Petrov et al., Published PCT Application WO 02/02714, and organometallic complexes described in, for example, published applications US 2001/0019782, EP 1191612, WO 02/15645, and EP 1191614; and mixtures thereof. Electroluminescent emissive layers comprising a charge carrying host material and a metal complex have been described by Thompson et al., in U.S. Pat. No. 6,303,238, and by Burrows and Thompson in published PCT applications WO 00/70655 and WO 01/41512. Examples of conjugated polymers include, but are not limited to poly(phenylenevinylenes), polyfluorenes, poly(spirobifluorenes), polythiophenes, poly(p-phenylenes), copolymers thereof, and mixtures thereof.

In one embodiment of the devices of the invention, photoactive material can be an organometallic complex. In another embodiment, the photoactive material is a cyclometalated complex of iridium or platinum. Other useful photoactive materials may be employed as well. Complexes of iridium with phenylpyridine, phenylquinoline, or phenylpyrimidine ligands have been disclosed as electroluminescent compounds in Petrov et al., Published PCT Application WO 02/02714. Other organometallic complexes have been described in, for example, published applications US 2001/0019782, EP 1191612, WO 02/15645, and EP 1191614. Electroluminescent devices with an active layer of polyvinyl carbazole (PVK) doped with metallic complexes of iridium have been described by Burrows and Thompson in published PCT applications WO 00/70655 and WO 01/41512. Electroluminescent emissive layers comprising a charge carrying host material and a phosphorescent platinum complex have been described by Thompson et al., in U.S. Pat. No. 6,303,238, Bradley et al., in Synth. Met. 2001, 116 (1-3), 379-383, and Campbell et al., in Phys. Rev. B, Vol. 65 085210.

A second electrical contact layer 160 is deposited on the organic layer 130. For illustrative purposes, the layer 160 is a cathode layer.

Cathode layers may be deposited as lines or as a film. The cathode can be any metal or nonmetal having a lower work function than the anode. Exemplary materials for the cathode can include alkali metals, especially lithium, the Group 2 (alkaline earth) metals, the Group 12 metals, including the rare earth elements and lanthanides, and the actinides. Materials such as aluminum, indium, calcium, barium, samarium and magnesium, as well as combinations, can be used. Lithium-containing and other compounds, such as LiF and Li₂O, may also be deposited between an organic layer and the cathode layer to lower the operating voltage of the system.

An electron transport layer 140 or electron injection layer 150 is optionally disposed adjacent to the cathode, the cathode being sometimes referred to as the “electron-injecting contact layer.”

An encapsulation layer 170 is deposited over the contact layer 160 to prevent entry of undesirable components, such as water and oxygen, into the device 100. Such components can have a deleterious effect on the organic layer 130. In one embodiment, the encapsulation layer 170 is a barrier layer or film.

Though not depicted, it is understood that the device 100 may comprise additional layers. For example, there can be a layer (not shown) between the anode 110 and hole transport layer 120 to facilitate positive charge transport and/or band-gap matching of the layers, or to function as a protective layer. Other layers that are known in the art or otherwise may be used. In addition, any of the above-described layers may comprise two or more sub-layers or may form a laminar structure. Alternatively, some or all of anode layer 110 the hole transport layer 120, the electron transport layers 140 and 150, cathode layer 160, and other layers may be treated, especially surface treated, to increase charge carrier transport efficiency or other physical properties of the devices. The choice of materials for each of the component layers is preferably determined by balancing the goals of providing a device with high device efficiency with device operational lifetime considerations, fabrication time and complexity factors and other considerations appreciated by persons skilled in the art. It will be appreciated that determining optimal components, component configurations, and compositional identities would be routine to those of ordinary skill of in the art.

In one embodiment, the different layers have the following range of thicknesses: anode 110, 500-5000 Å, in one embodiment 1000-2000 Å; hole transport layer 120, 50-2000 Å, in one embodiment 200-1000 Å; photoactive layer 130, 10-2000 Å, in one embodiment 100-1000 Å; layers 140 and 150, 50-2000 Å, in one embodiment 100-1000 Å; cathode 160, 200-10000 Å, in one embodiment 300-5000 Å. The location of the electron-hole recombination zone in the device, and thus the emission spectrum of the device, can be affected by the relative thickness of each layer. Thus the thickness of the electron-transport layer should be chosen so that the electron-hole recombination zone is in the light-emitting layer. The desired ratio of layer thicknesses will depend on the exact nature of the materials used.

In operation, a voltage from an appropriate power supply (not depicted) is applied to the device 100. Current therefore passes across the layers of the device 100. Electrons enter the organic polymer layer, releasing photons. In some OLEDs, called active matrix OLED displays, individual deposits of photoactive organic films may be independently excited by the passage of current, leading to individual pixels of light emission. In some OLEDs, called passive matrix OLED displays, deposits of photoactive organic films may be excited by rows and columns of electrical contact layers.

Devices can be prepared employing a variety of techniques. These include, by way of non-limiting exemplification, vapor deposition techniques and liquid deposition. Devices may also be sub-assembled into separate articles of manufacture that can then be combined to form the device.

### DEFINITIONS

The use of “a” or “an” are employed to describe elements and components of the invention. This is done merely for convenience and to give a general sense of the invention. This description should be read to include one or at least one and the singular also includes the plural unless it is obvious that it is meant otherwise.

The term “active” when referring to a layer or material is intended to mean a layer or material that exhibits electronic or electro-radiative properties. An active layer material may emit radiation or exhibit a change in concentration of electron-hole pairs when receiving radiation. Thus, the term “active material” refers to a material which electronically facilitates the operation of the device. Examples of active materials include, but are not limited to, materials which conduct, inject, transport, or block a charge, where the charge can be either an electron or a hole. Examples of inactive materials include, but are not limited to, planarization materials, insulating materials, and environmental barrier materials.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The term “layer” is used interchangeably with the term “film” and refers to a coating covering a desired area. The area can be as large as an entire device or a specific functional area such as the actual visual display, or as small as a single sub-pixel. Films can be formed by any conventional deposition technique, including vapor deposition and liquid deposition. Liquid deposition techniques include, but are not limited to, continuous deposition techniques such as spin coating, gravure coating, curtain coating, dip coating, slot-die coating, spray-coating, and continuous nozzle coating; and discontinuous deposition techniques such as ink jet printing, gravure printing, and screen printing.

The term “organic electronic device” is intended to mean a device including one or more semiconductor layers or materials. Organic electronic devices include, but are not limited to: (1) devices that convert electrical energy into radiation (e.g., a light-emitting diode, light emitting diode display, diode laser, or lighting panel), (2) devices that detect signals through electronic processes (e.g., photodetectors photoconductive cells, photoresistors, photoswitches, phototransistors, phototubes, infrared (“IR”) detectors, or biosensors), (3) devices that convert radiation into electrical energy (e.g., a photovoltaic device or solar cell), and (4) devices that include one or more electronic components that include one or more organic semiconductor layers (e.g., a transistor or diode). The term device also includes coating materials for memory storage devices, antistatic films, biosensors, electrochromic devices, solid electrolyte capacitors, energy storage devices such as a rechargeable battery, and electromagnetic shielding applications.

The term “substrate” is intended to mean a workpiece that can be either rigid or flexible and may include one or more layers of one or more materials, which can include, but are not limited to, glass, polymer, metal, or ceramic materials, or combinations thereof.

As described herein, a monomeric unit derived from a monomer refers to the unit formed when the polymerizable group is polymerized. For example, when the polymerizable group is a vinyl group, polymer having repeating units derived from a monomer

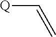

will have repeating units of

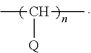

The term “reactive group” is intended to mean a group that is capable of reacting to lead to further polymerization or crosslinking of the initial polymer chains.

Unless otherwise defined, all technical and scientific terms used herein have the same meaning as commonly understood by one of ordinary skill in the art to which this invention belongs. Although methods and materials similar or equivalent to those described herein can be used in the practice or testing of embodiments of the present invention, suitable methods and materials are described below. All publications, patent applications, patents, and other references mentioned herein are incorporated by reference in their entirety, unless a particular passage is cited. In case of conflict, the present specification, including definitions, will control. In addition, the materials, methods, and examples are illustrative only and not intended to be limiting.

To the extent not described herein, many details regarding specific materials, processing acts, and circuits are conventional and may be found in textbooks and other sources within the organic light-emitting diode display, photodetector, photovoltaic, and semiconductive member arts.

### EXAMPLES

The concepts described herein will be further described in the following examples, which do not limit the scope of the invention described in the claims.

### Example 1

A suspension of Pd₂(dba)₃ (1.48 g, 1.61 mmol) and 2-(dicyclohexylphosphino)biphenyl (1.36 g, 3.87 mmol) in THF (30 mL) is stirred for 30 minutes. To this, 4-bromo-4′-hydroxybiphenyl (8.04 g, 32.3 mmol), N,N′-diphenylbenzidine (22.1 g, 65.6 mmol) and LiN(Si(Me)₃)₂ (11.89 g, 71.0 mmol) in THF (70 mL) is added and the resulting mixture is refluxed until complete conversion to compound 1 is achieved. Upon cooling to room temperature, 1M HCl (˜100 mL) is added and allowed to stir for 10 minutes followed by neutralization with saturated NaHCO₃ solution. The organic layer is separated, washed with brine and dried over magnesium sulfate. Upon evaporation, product 1 is purified by chromatography.

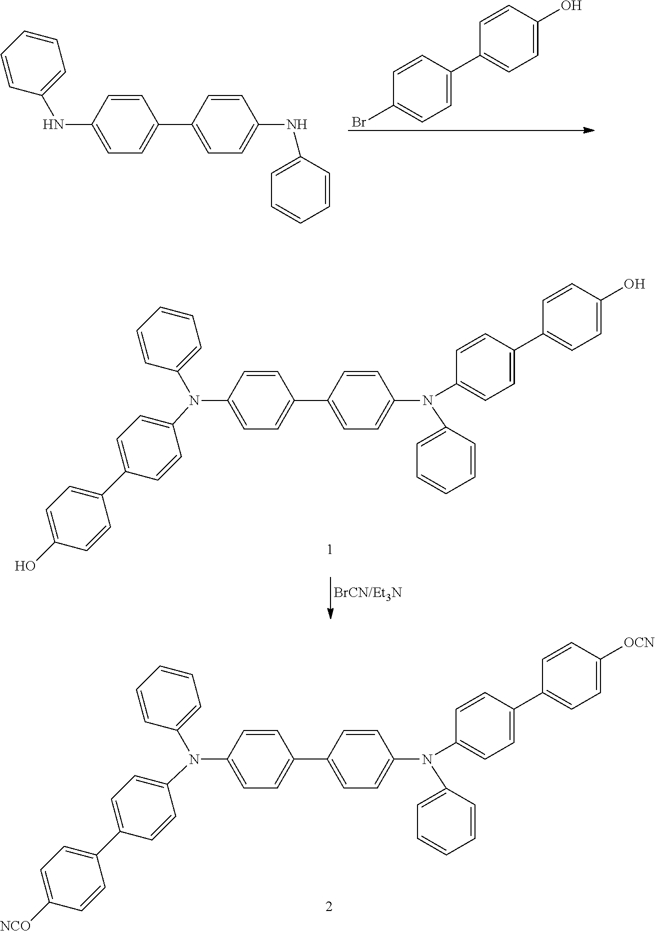

### Example 2

### Synthesis of Compound 2

Compound 1 (1.00 g, 1.48 mmol) is dissolved in CH₂Cl₂ and mixed with cyanogen bromide (0.4 g). After cooling (−20° C.) the resulting mixture, a CH₂Cl₂ solution of Et₃N (0.45 g) is added. The mixture is then allowed to slowly warm up to room temperature. After addition of 1M HCl, the organic layer is separated and dried over MgSO₄. Removal of solvent and purification using chromatography provides compound 2.

### Example 3a

### Copolymer of 4-vinyl-triphenylamine and 3-vinylbenzaldehyde

Take 2 g (0.0072 moles)4-vinyl-triphenylamine and 0.18 g 3-vinylbenzaldehyde (0.0014 moles) and dissolve into 7 mL toluene under nitrogen in a glove box. Add 20 mg AIBN and stir and warm to 70° C. overnight. Take the pale yellow solution and pour quickly into 25 mL methanol with stirring. Filter off the white sticky solid and suction dry to a white powder. Redissolve into methylene chloride and add hexanes to stirred solution to re-precipitate a white solid. Collect by filtration and suction dry to white solid again. Wash with methanol and hexanes

1-H nmr in methylene chloride looks good with good incorporation of the aldehyde as judged by the peak at 9.8 ppm:

### Example 3b

### Conversion of Co-Polymer 1a to a Crosslinkable Version by Transformation of Aldehyde Groups to Vinyl Groups

Dissolve 0.356 g methyltriphenylphosphonium bromide into 2 mL THF and add 0.096 g sodium t-butoxide with cooling. Stir for 30 mins then add 1.22 g of polymer 1a and the solution turns orange and gets warm. Stir for 5 hrs at RT then let sit at room temperature overnight. Evaporate in a nitrogen stream and then wash with hexanes and methanol and extract into methylene chloride. Filter through celite to give a pale yellow solution which was evaporated and then precipitated with methanol to give a powdery white solid (recover ˜700 mg). 1-H nmr clearly shows loss of the aldehyde and concomitant appearance of vinyl protons:

DSC of polymer 3b reveals a Tg of ˜136° C. and an exotherm at ˜240° C. Resulting film is light cream colored and largely insoluble in toluene and methylene chloride.

### Example 4a

### Copolymer of N-(3-vinylphenyl), N-(1-naphthyl)-aniline, and 3-vinylbenzaldehyde

Take 5 g N-(3-vinylphenyl), N-(1-naphthyl)-aniline (15.5 mM) and 1.25 g 3-vinylbenzaldehyde (9.5 mM) and dissolve into 3 mL chlorobenzene in a brown bottle in a nitrogen filled glove box. Add 20 mg AIBN and stir and warm to 70° C. Heat and stir over night. The vial becomes filled with thick clear liquid. Remove from nitrogen glove box and add methanol to precipitate a powdery white solid. Filter off methanol and dissolve solid into methylene chloride as a clear thick liquid. Stir and add hexanes to reprecipitate a white solid. Collect by filtration and dry in nitrogen stream.

1-H nmr in methylene chloride shows that the product still contains a lot of chlorobenzene as well as methanol, toluene and hexanes but otherwise looks good—no vinyls left and approximately correct level of aldehyde (30:1 proton ratio) which should lead to 38% crosslink density:

### Example 4b

### Conversion of Polymer 4a to Crosslinkable Version Via Transformation of Aldehyde Groups to Vinyl Groups

Dissolve 4 g methyltriphenylphosphonium bromide into 25 mL THF and add 1.1 g sodium t-butoxide with cooling. Stir for 30 mins then add 5 g of polymer 2a in THF—turns very pale orangey red and gets warm. Stir for 5 hrs at room temperature then let sit at room temperature overnight. Evaporate in nitrogen stream and then wash with water and then methanol to generate a powdery white solid. Collect on frit and wash well with methanol and hexanes then suction dry. Redissolve into methylene chloride and filter through a short silica gel plug. Evaporate and wash well with methanol and hexanes to isolate a white powder. Suction dry and redissolve into methylene chloride and chromatograph on silica eluting with methylene chloride which removes traces of phosphorus containing impurities. 1-H nmr spectral integrals show the ratio of monomers in the polymer is almost exactly 2.2:1 triarylamine:vinyl and with no residual aldehyde functionality.

DSC of polymer 4b reveals a Tg of ˜134° C. and a crosslinking exotherm at ˜220° C. Resulting film is light cream colored and largely insoluble in toluene and methylene chloride.

### Example 5

### Synthesis of a Monomer of

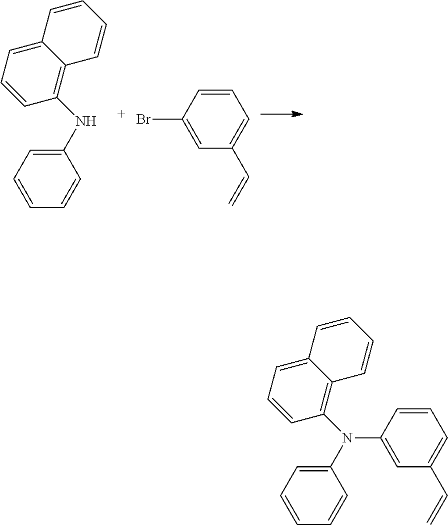

A mixture of phenyl(1-naphthyl)amine (10 g, 45.0 mmol), 3-bromostyrene (9.2 g, 50.0 mmol), NaO^{t}Bu (5.1 g, 55.0 mmol), Pd₂(dba)₃ (0.300 g, 0.33 mmol) and P(^{t}Bu)₃ (0.150 g, 0.74 mmol) was stirred in toluene (80 mL) under nitrogen for 20 hours. The resulting solution was diluted with diethylether and filtered through celite and silica. Upon evaporation of the solvent a dark brown viscous material was obtained which was purified by chromatography on silica (hexanes) and the desired product was obtained as a white solid (9.8 g, 67%). The product's ¹H NMR is shown below.

### Example 6

### Synthesis of a Monomer of

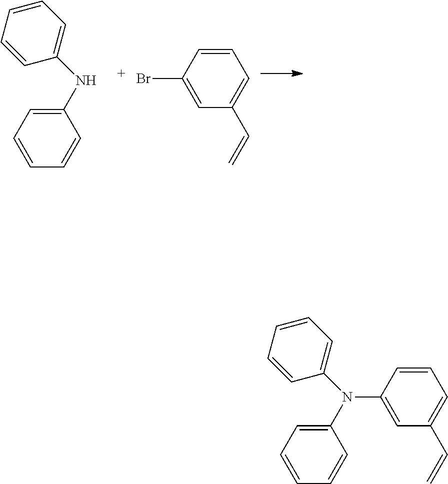

A mixture of diphenylamine (4.6 g, 27.0 mmol), 3-bromostyrene (5.0 g, 27.0 mmol), NaO^{t}Bu (3.0 g, 38.0 mmol), Pd₂(dba)₃ (0.300 g, 0.33 mmol) and P(^{t}Bu)₃ (0.150 g, 0.74 mmol) was stirred in toluene (80 mL) under nitrogen for 20 hours. The resulting solution was diluted with diethylether and filtered through celite and silica. Upon evaporation of the solvent a dark brown viscous material was obtained which was purified by chromatography on silica (hexanes) and the desired product was obtained as a white solid (4.25 g, 65% yield). The product's ¹H NMR is shown below.

### Example 7

### Synthesis of a Monomer of

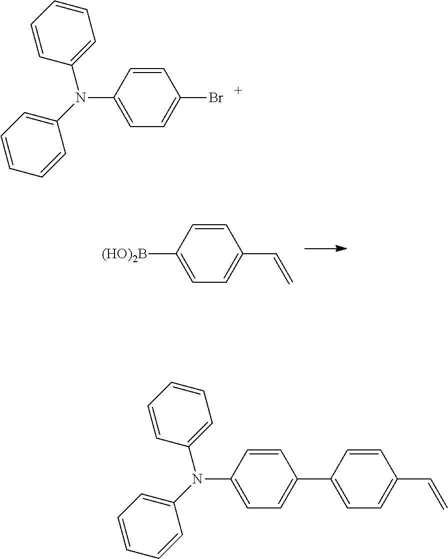

A degassed solution of K2CO3 (8.20 g, 15.4 mmol) in H2O (100 mL) was added to a mixture of (4-bromophenyl)diphenyl amine (5.00 g, 1.54 mmol), 4-(vinyl)phenylboronic acid (3.53 g, 18.2 mmol) and Pd(PPh3)₄ (0.89 g, 0.77 mmol) in monoglyme (100 mL) and then heated to 80° C. overnight. Upon cooling, the mixture was diluted with diethylether and 1M HCl (˜10 mL) was added. After neutralization with a saturated solution of NaHCO₃, the organic layer was separated and dried over MgSO₄. Upon evaporation of the solvent a yellow solid was obtained which was purified by chromatography on silica (hexane) to obtain the desired product as a white powder (2.2 g, 41%). The product's ¹H NMR is shown below.

### Example 8

### Synthesis of a Monomer of

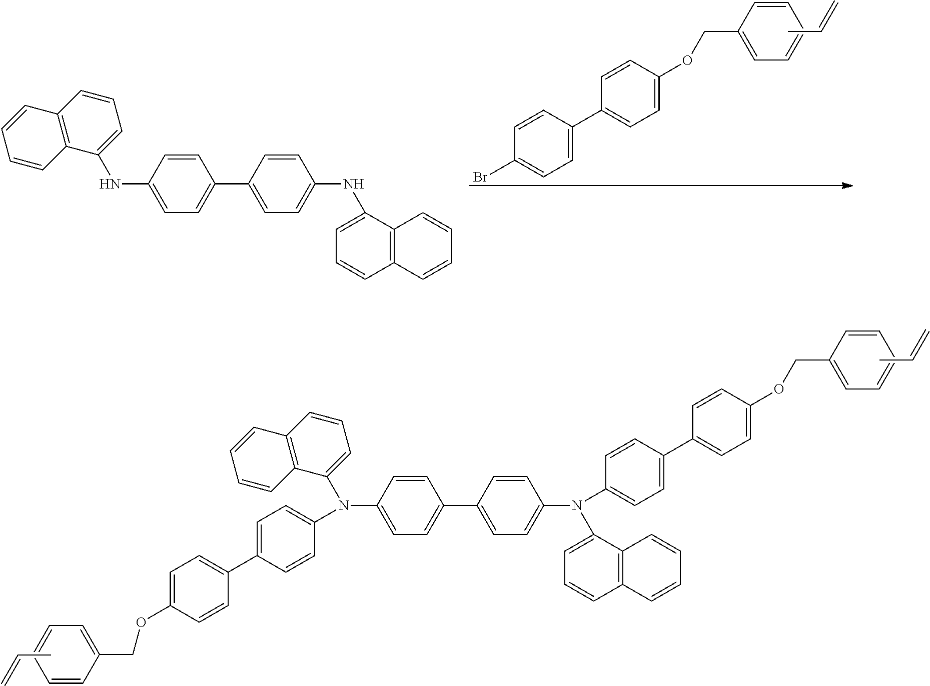

A mixture of N,N′-di(1-naphthyl)benzidine (2.16 g, 4.9 mmol), 4-bromo-1,1′-biphenyl-4′-(p,m-vinyl)benzyl (3.8 g, 10 mmol), NaO^{t}Bu (1.15 g, 12 mmol), Pd₂(dba)₃ (0.300 g, 0.33 mmol) and P(^{t}Bu)₃ (0.150 g, 0.74 mmol) was stirred in toluene (30 mL) under nitrogen for 20 hours. The resulting solution was diluted with diethylether and filtered through celite and silica. Upon evaporation of the solvent a dark brown viscous material was obtained. Addition of hexane produced a yellow powder, which was isolated by filtration. Further purification from CH₂Cl₂/hexane gave the desired product (1.63 g, 33%). The product's ¹H NMR is shown below.

### Example 9

### Synthesis of a Monomer of

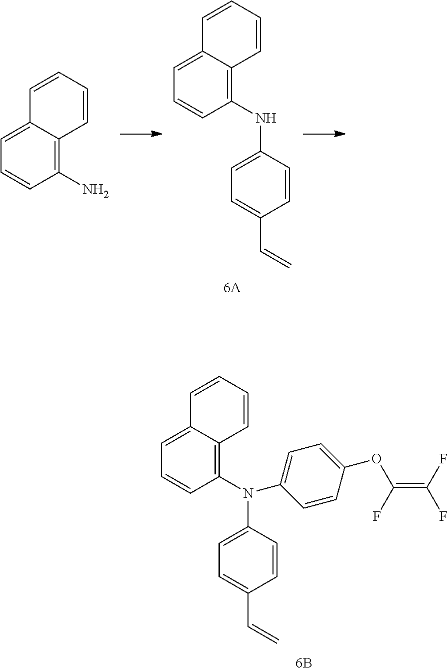

Synthesis of compound 6A: A mixture of 1-naphthylamine (5.00 g, 34.9 mmol), 4-bromo-styrene (4.6 g, 25.1 mmol), NaO^{t}Bu (2.55 g, 27.4 mmol), Pd₂(dba)₃ (0.300 g, 0.33 mmol) and P(^{t}Bu)₃ (0.150 g, 0.74 mmol) was stirred in toluene (30 mL) under nitrogen for 20 hours. The resulting solution was diluted with diethylether and filtered through celite and silica. Upon evaporation of the solvent a dark brown viscous material was obtained. Addition of hexane produced a yellow powder, which was isolated by filtration. Further purification from CH₂Cl₂/hexane gave the desired product as a white powder (0.9 g, 10%).

Synthesis of p-(N-1-Naphthyl-N′-4-perfluorovinyloxyphenyl)aminostyrene (6B): A mixture of 6A (1.07 g, 4.36 mmol), 1-bromo-4-trifluorovinyloxybenzene (1.38 g, 5.45 mmol), (^{t}Bu₃P)₂Pd (56 mg, 0.11 mmol) and sodium t-butoxide (0.42 g, 4.4 mmol) is stirred in toluene (9 mL) in a N₂ purged glovebox for 16 hours. The resulting mixture is diluted with diethyl ether (90 mL) and filtered through Celite®. The solution is concentrated on a rotary evaporator and further dried under high vacuum to yield a dark brown oil. The product is purified by flash column chromatography (silica gel; 12:1 hexanes/CH₂Cl₂) to yield a clear oil (1.4 g, 77%). ¹H NMR (500 MHz, CDCl₃, TMS) spectrum was consistent with the desired product.

### Example 10

### Copolymer of N-(4-vinylphenyl),N-(1-naphthyl)(4-perfluorovinyloxyaniline) and N-(3-vinylphenyl), N-(1-naphthyl)aniline

Take 1.35 g monomer 6b above with 2.6 g monomer from example 5 and dissolve into 3 mL toluene in a brown vial in a nitrogen filled glove box. 20 mg AIBN was added and the mixture stirred at 70° C. The mix was heated and stirred overnight. Next day, the vial contained a thick clear liquid. The vial was removed from the glovebox and methanol was added to ppt a thick gooey white solid. The methanol was decanted and the white paste was dissolved into methylene chloride as a clear thick liquid. Hexanes was added to ppt again a white paste which was collected by decanting solvent and drying in a nitrogen stream. A final reprecipitation from toluene by addition of methanol gave a powdery pale cream polymer product

19-F and 1-H nmr spectra are consistent with the expected product containing small amounts of unreacted vinyl monomer as well as toluene solvent

### Example 11

Polymer of p-(N-1-Naphthyl-N′-4-perfluorovinyloxyphenyl)aminostyrene:

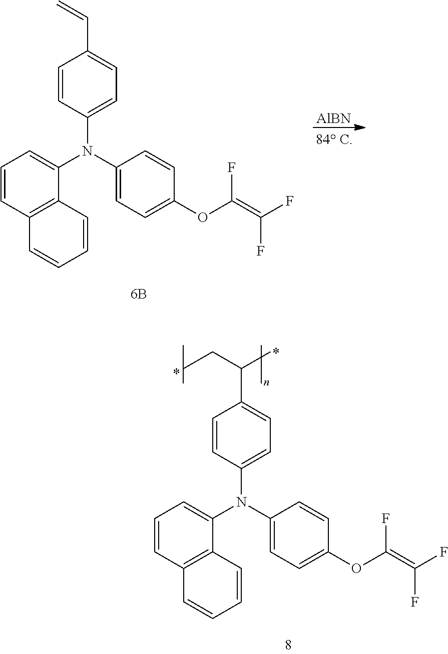

A 25 mL Schlenk tube is charged with a mixture of 6B (0.52 g, 1.3 mmol), AIBN (5.5 mg, 1.0 wt %) and toluene (0.6 g) in a N₂ purged glovebox. The solution is heated at 84° C. for 23 hours in a heated aluminum block and then cooled to room temperature. The polymer solution is diluted with toluene (10 mL) and precipitated once from acetone:MeOH (1:1, 125 mL) and then redissolved in THF (2 mL) and precipitated in MeOH (75 mL). After drying under high vacuum, the desired polymer is obtained as a cream colored solid (370 mg, 71.0%). The molecular weight of the resulting polymer measured by Size Exclusion Chromatography (THF, vs. polystyrene standards) is M_{w}=31,800; Mₙ=8,600 and M_{w}/Mₙ=3.70. ¹H NMR (500 MHz, CDCl₃, TMS) spectrum was consistent with the desired product.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

Many aspects and embodiments have been described above and are merely exemplary and not limiting. After reading this specification, skilled artisans appreciate that other aspects and embodiments are possible without departing from the scope of the invention.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

It is to be appreciated that certain features are, for clarity, described herein in the context of separate embodiments, may also be provided in combination in a single embodiment. Conversely, various features that are, for brevity, described in the context of a single embodiment, may also be provided separately or in any subcombination. Further, reference to values stated in ranges include each and every value within that range.

1. A compound of formulae I or II: 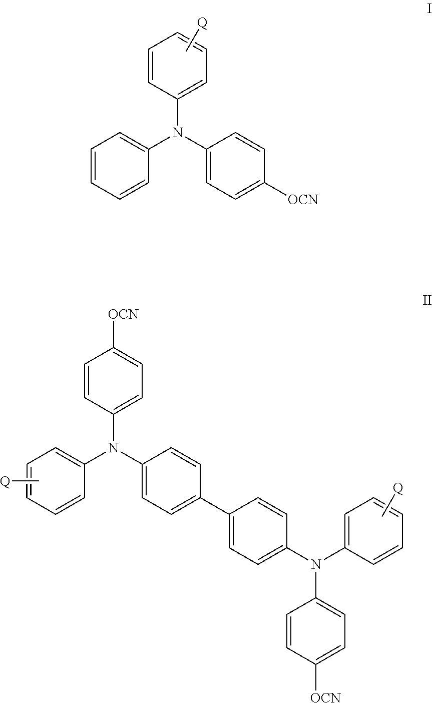
wherein:
Q is, independently at each occurrence, a vinyl group, an acrylate group, or a methacrylate group.

2. The compound of claim 1, wherein Q is in the para position.

3. The compound of claim 1, wherein Q is a vinyl group.

4. A polymer comprising units formed from at least one of formulae I or II of the compound of claim 1.

5. The polymer of claim 4, wherein the polymer is a random, block, graft, or alternating copolymer.

6. The polymer of claim 4, having formula III:

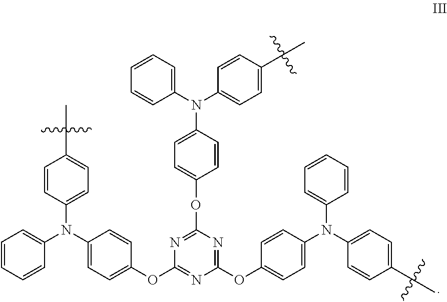

7. The polymer of claim 4, having formula IV:

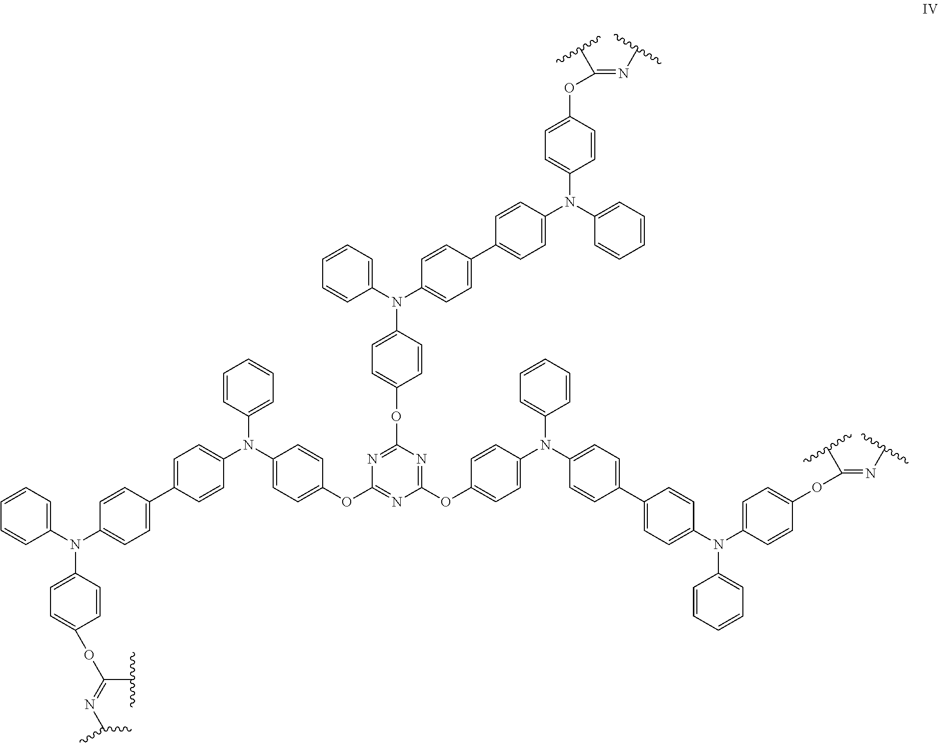

8. A composition including the compound of claim 1 or the polymer of claim 4.

9. An organic electronic device having an active layer including the polymer of claim 4.

10. An article useful in the manufacture of an organic electronic device, comprising the polymer of claim 4.

11. A compound of formula VI: 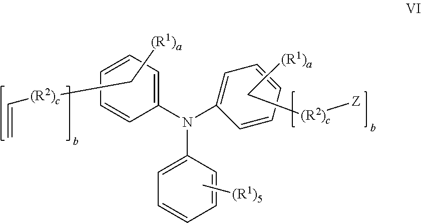
wherein:
R¹ can be the same or different at each occurrence and is selected from H, D, alkyl, heteroalkyl, aryl, heteroaryl, arylalkylene, heteroarylalkylene, CₙH_{d}Fₑ, C₆H_{f}F_{g}, arylamine, arylalkylamine, arylether, alkylether, arythioether and alkylthioether;
R² can be the same or different at each occurrence and is selected from alkyl, heteroalkyl, aryl, heteroaryl, arylalkylene, heteroarylalkylene, CₙHₕF_{I}, C₆HⱼFₖ, arylamine, arylalkylamine, arylether, alkylether, arythioether and alkylthioether;
Adjacent R¹ and/or R² can be joined to form a a fused alkyl or aromatic five or six membered ring;
a is 0 or an integer from 1 through 4 and b is an integer from 1 through 5 such that a+b<=5;
c is 0 or an integer from 1 through 20;
d, e, f and g are an integer such that e+f=2n+1, and g+h=5;
h, i, j and k are an integer such that h+i=2n, and j+k=4;
n is an integer 1 through 20; and
Z is a second substituent having a reactive group.

12. The compound of claim 11, wherein the reactive group is a cyanate group, a vinyl group, a perfluorovinyl ether group, 3,4-benzocyclobutan-1-yl group, a siloxane group, or an aldehyde group.

13. The compound of claim 11, wherein the reactive group is a group which can further react to give a polymerizable group.

14. A polymer comprising at least one monomeric unit derived a compound of formulae XIV, XV, or mixtures thereof: 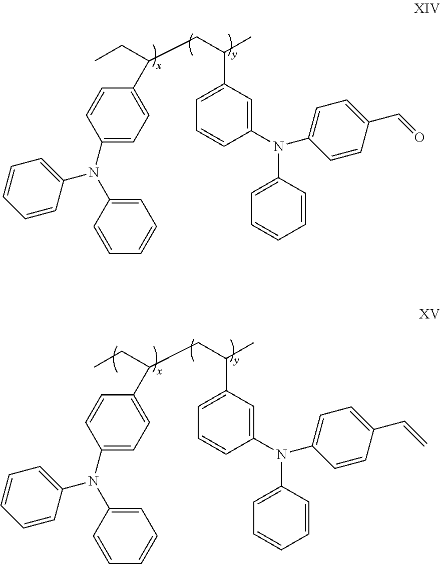
wherein x and y are integers equal to or greater than 1.
